(19) United States
(12) Patent Application Publication
(10) Pub. No.: US 2013/0101536 A1
(43) Pub. Date: Apr. 25, 2013

(54) Odor Reduction Compositions

(71) Applicant: Quest International Services B.V., Naarden (NL)

(72) Inventors: Keith Douglas Perring, Kent (GB); Anne Richardson, Kent (GB); Christine Margaret Goulding, Kent (GB); Jean-Pascal Osmont, Saint Pierre de Salerne (FR)

(73) Assignee: Quest International Services B.V., Naarden (NL)

(21) Appl. No.: 13/714,057

(22) Filed: Dec. 13, 2012

Related U.S. Application Data
(63) Continuation of application No. 11/596,023, filed on Apr. 14, 2008, now abandoned.

(30) Foreign Application Priority Data
May 12, 2004 (GB) 0410583.9
Feb. 26, 2005 (GB) 0504003.5

(51) Int. Cl. A61K 8/49; A61K 8/37; A61K 8/33; A61Q 5/06; A61Q 5/08

(52) U.S. Cl. CPC A61K 8/498; A61Q 5/065; A61Q 5/08; A61K 8/33; A61K 8/37
USPC 424/62; 424/70.6; 424/70.1

(57) ABSTRACT

An odor-reducing composition for counteracting malodour in which the composition includes an odor reducing material of the type phenyl ethyl methyl ether, Cyprisate, Camonal and paracresyl methyl ether and a method for counteracting malodour employing such a composition.

### FIELD OF INVENTION

This invention is concerned with fragrance compositions that reduce the sensory perception of malodorous amines. More particularly, this invention is directed to fragrance compositions comprising certain fragrance ingredients that reduce the sensory perception of malodorous amines while maintaining their fragrance intensity.

### BACKGROUND

There are numerous sources of malodorous amines and a persistent problem is the pungent odor that is produced by them.

Typical sources of malodorous amines include landfills, cat litter, chicken coops, water treatment plants and ponds, garbage, dog kennels, rendering plants, food processing plants, wool plants, fish canneries, sewers, paper mills and rest rooms.

Typical examples of products containing malodorous amines are hair-treatment products such as hair bleaches and colorants.

Hair bleaching and coloring compositions normally contain oxidative agents such as inorganic peroxygen oxidizing agents, and they may additionally contain peroxide activating agents such as aqueous hydrogen peroxide solutions.

Unfortunately, the action of hydrogen peroxide solutions is slow and their use in the treatment of hair may solubilize and decolorize melanin leading to hair damage and undesirable qualities such as brittleness. Also, in order to maintain the necessary level of activity for the aqueous hydrogen peroxide solution, it is necessary that the final hair treatment composition has a pH of from about 9 to about 12. Below a pH of 9, insufficient bleaching action occurs; above pH 12 an excessive amount of hair damage occurs.

To minimize damage to the hair and to increase the reaction rate, an activator is normally used. Ammonia is known to be an effective activator of the hair-bleaching action of aqueous peroxide solutions. It operates by accelerating the oxidative destruction of particles of hair pigment while its pH adjusting properties maintain the required alkalinity. Therefore, it has become common practice to use ammonia both for its activator effect and for its pH adjusting effect. As a result, the vast majority of hair-bleaching and/or coloring products contain substantial quantities of ammonia.

Unfortunately, the inclusion of ammonia and its derivatives (such as alkanolamines)in hair bleaching and/or coloring products introduces a strong, offensive and difficult to mask malodorous amine odor and provides a hostile environment within which it is difficult to achieve effective perfuming.

Therefore, there is a need for stable compositions to suppress malodorous amines, particularly the odors associated with ammonia in hair-treatment products.

European Patent Specification No. 1133982 describes perfume materials containing a phenyl ring moiety having an air diffusion coefficient of >5.7 or a C-5 ring moiety, which also contains at least 1 carbon which is sp2 hybridized having an air-diffusion coefficient of >4.4 for use in ammonia-containing products.

WO 00/37117 describes various compositions having a reduced malodour which contain, inter alia 5-methyl-2-(2-methylpropyl)-1,3-dioxane (hereinafter referred to as “Camonal”) or methyl 1,4-dimethylcyclohexylcarboxylate (hereinafter referred to as “Cyprisate”) as fragrance materials.

### SUMMARY

According to the invention there is provided an odor-reducing composition for counteracting malodorous amines comprising at least one odor-reducing material selected from the group consisting of phenyl ethyl methyl ether, Cyprisate, Camonal and paracresyl methyl ether.

Preferably, the odor-reducing composition comprises at least two odor-reducing materials selected from the group consisting of phenyl ethyl methyl ether, Cyprisate, Camonal and paracresyl methyl ether.

Suitably, the odor reducing material is present at 10 weight % or above. Advantageously, the odor reducing material is present at 15 weight % or above. More preferably, the odor reducing material is present at 30 weight % or above.

The invention also extends to a hair treatment composition comprising an odor-reducing composition as previously defined.

Preferably, the composition is a hair colorant. Alternatively, the composition is a bleaching composition for hair.

In one embodiment of the invention, the malodorous amine comprises ammonia. Suitably, the ammonia is present in an amount from 0.5 weight % to 5 weight % in the hair treatment composition.

According to the invention there is also provided a method of counteracting malodorous amines comprising adding to the source of the malodor a malodor counteracting amount of an odor-reducing composition comprising at least one odor-reducing material selected from the group consisting of phenyl ethyl methyl ether, Cyprisate, Camonal and paracresyl methyl ether.

Preferably, the malodour counteracting amount of the odor-reducing composition comprises at least two odor-reducing materials selected from the group consisting of phenyl ethyl methyl ether, Cyprisate, Camonal and paracresyl methyl ether.

Suitably the odor reducing material is present in said odor-reducing composition at 10 weight % or above. Preferably, the odor reducing material is present in said odor-reducing composition at 15 weight % or above. More preferably, the odor reducing material is present in said odor-reducing composition at 30 weight % or above.

Advantageously, the malodorous amine comprises ammonia in the method of the invention. In one embodiment, the source of the malodour comprises a hair-treatment product. Suitably, the hair-treatment product is a hair colorant. Alternatively, the hair-treatment product is a bleaching composition for hair.

The present invention relates to compositions for reducing the perception of malodorous amines, wherein such composition comprises at least one odor-reducing material selected from the group consisting of Phenyl ethyl methyl ether, Cyprisate, Camonal and Paracresyl methyl ether.

Although it is known to include Phenyl ethyl methyl ether, Cyprisate, Camonal and Paracresyl methyl ether in fragrance compositions, it was not known that their inclusion in compositions for use in environments containing malodorous amines could impart odor-counteracting properties. In particular, it has been found that the odor-reducing materials are olfactively stable in environments containing relatively high ammonia concentrations such as hair colorants and bleaches.

Moreover, surprisingly, it has now been found that an odor-reducing composition for counteracting malodorous amines comprising at least two odor-reducing materials selected from the group comprising phenyl ethyl methyl ether, Cyprisate, Camonal and paracresyl methyl ether is particularly efficacious.

### DETAILED DESCRIPTION

With the intention of counteracting the unpleasant smells associated with malodorous amines, such as the smell of ammonia from ammonia-containing hair-treatment products, an investigation was carried out into the effectiveness of a variety of fragrance materials.

The following fragrance materials were tested at the dilutions (v/v) shown. The perceived intensity of each fragrance material was measured, and the perceived intensity of the combination of each fragrance material and malodorous amine was measured in an enclosed environment.
Allyl amyl glycolate (Q) 1%
Allyl heptanoate 1%
Allyl hexanoate 1%
Amyl salicylate 20%
Benzyl salicylate 20%
Camonal 5%
Cis 3 Hexenol (Green Grass) 10%
Citronellol pure 10%
Citronellyl propionate 5%
Cressanther 3%
Cyprisate 2%
Diphenyl oxide 1%
Dispirone 0.5%
Elintaal 15%
Ethyl hepanoate 1%
Ethyl methyl phenyl glycidate 1%
Ethylene brassylate 20%
Florocyclene 5%
Galaxolide solvent free 20%
Gardocyclene 5%
Geranyl propionate 10%
Hexyl salicylate 10%
Iso eugenyl acetate 0.5%
Lilial 10%
Lixetone (Q) 5%
Methyl benzoate 1%
Methyl ionone alpha iso (Q) 5%
Methyl salicylate 1%
Nonalactone gamma 1%
Paracresyl methyl ether 1%
Petiole 1%
Phenoxyethyl isobutyrate beta 5%
Phenyl ethyl methyl ether 3%
Phenyl ethyl phenylacetate 10%
Phenyl ethyl salicylate 5%
Styrallyl propionate 2%
Tetrahydro geraniol 5%
Tetrahydro myrcenol 5%
Timberol 1%
Undecalactone gamma 1%

To determine the performance of each fragrance ingredient against malodorous amines, the following protocols were used.

It is to be noted that evaluations are carried out by 18 to 24 panelists who have been extensively screened for their olfactory acuity and trained in the methods of evaluation.

### Ingredient Intensity: Evaluation Protocol

Intensity was measured by placing a 1 ml sample of fragrance material in a 60 ml bottle and having it rated by a trained sensory panel. Each fragrance material was assessed 20 times.

### Ingredient Performance Against Malodorous Amine: Evaluation Protocol

Performance was evaluated by placing a 3 ml sample of malodor (0.1N ammonium hydroxide) in an uncapped 15 ml wide-mouth jar. This jar was then placed into a 500 ml wide-mouth jar containing 1 ml of test fragrance material in an uncapped 15 ml wide-mouth jar. The 500 ml jar was then capped and the system was left to equilibrate for 30 minutes.

A trained sensory analysis panel using an established scaling technique measured the perceived intensity of ammonia malodor and fragrance material in each sample.

In individual panel sessions, each panelist assessed four fragrance materials. Each fragrance material was assessed against a malodor control consisting of a 500 ml wide-mouth jar containing a 15 ml wide-mouth jar containing 3 ml (0.1N) ammonium hydroxide and a 15 ml wide-mouth jar containing 1 ml DEP. A hidden control was also included with the samples in order to check the consistency of scaling. To eliminate the build up of ammonia in the jar headspace, panelists were asked to open the jar and wait 3 seconds before beginning the assessment. A 5 minute rest period was given between each fragrance material in order to minimize fatigue and to adhere to recommended safety standards.

A total of 20 assessments were made of each sample. The results of the assessments were analyzed using Analysis of Variance (ANOVA) and multiple comparison 1.3 tests.

The single fragrance ingredient intensities, and the malodor intensities and fragrance ingredient intensities in the ingredient/malodor mix, are given in Table 1 below.

TABLE 1

| | Ingredient/malodor mix | | |
|---|---|---|---|
| | Malodor | Ingredient | Ingredient |
| Product | intensity | intensity | intensity |
| Phenyl ethyl methyl ether 3% | 29 | 53 | 67 |
| Cyprisate 2% | 34 | 54 | 55 |
| Camonal 5% | 35 | 56 | 66 |
| Paracresyl methyl ether 1% | 37 | 52 | 71 |
| Cis-3-hexenyl salicylate | 56 | 28 | 24 |

Cis-3-hexenyl salicylate was used as a benchmark reference.

Table 1 shows clearly that the fragrance materials Phenyl ethyl methyl ether, Cyprisate, Camonal and Paracresyl methyl ether act as odor-reducing materials, significantly reducing the perceived intensity of the malodor while retaining their ingredient intensity.

To achieve the highly efficacious odor counteracting and fragrance effect of this invention, it is particularly preferred that compositions for reducing the perception of malodorous amines comprise at least two odor-reducing material selected from the group consisting of Phenyl ethyl methyl ether, Cyprisate, Camonal and Paracresyl methyl ether.

More preferably the compositions for reducing the perception of malodorous amines will comprise at least 10 wt. % of odor reducing materials; even more preferably at least 15 wt. % of odor reducing materials; more preferably still at least 30 wt. of odor reducing materials.

The compositions for reducing the perception of malodorous amines can then be incorporated into malodorous compositions as required.

As used herein the term ‘hair’ to be treated may be ‘living’ (i.e. on a living body) or may be ‘non-living’ (i.e. in a wig, hairpiece or other aggregation of non-living fibers, such as is used in textiles and fabrics). Mammalian, especially human, hair is preferred. However, wool, fur and other melanin containing fibers are also included.

It is understood that the compositions and method of the present invention are not restricted to any particular physical mode or product form, and may be contained for example and not as the limitation to the present invention, in aqueous and non-aqueous products, foams, powders, granules, gels, aerosols, non-aerosols, waxes, microencapsulated vehicles, phase-change microencapsulated vehicles, and the like.

Compositions of the present invention may be used in a number of malodorous amine containing environments or products. For example, and not as a limitation to the present invention, environments such as land fills, cat litter, chicken coops, water treatment plants and ponds, garbage, dog kennels, rendering plants, food processing plants, wool plants, fish canneries, sewers, paper mills and rest rooms, and products for bathroom care, room freshening, air freshening, pet care, adult incontinence, household cleaning, hair treatment, hard surface cleaning, and the like.

### EXAMPLES

Examples of preferred odor-reduction compositions of the present invention are as follows:

### Example 1

| | % |
|---|---|
| Mandarin Italian Pure | 3.2 |
| Citronellyl nitrile (Q) | 0.8 |
| Tridecen-2-nitrile 10% dep (Q) | 0.4 |
| CYPRISATE CI (Q) | 40.0 |
| Cis-3-hexenyl acetate | 0.4 |
| Ligustral (Q) | 0.8 |
| Efetaal (Q) | 1.6 |
| Lilial | 4.0 |
| Iso Jasmone Pure (Q) | 0.4 |
| Phenyl ethyl alcohol | 36.0 |
| Maceal 10% dpg (Q) | 0.4 |
| Patchouli acid washed (Q) | 4.0 |
| Acetylcedrene (Q) | 4.8 |
| Sandela | 1.6 |
| Heliotropin | 1.6 |

### Example 2

| | % |
|---|---|
| Dihydromyrcenol (Q) | 10.0 |
| Herboxane (Q) | 10.0 |
| CAMONAL CI (Q) | 15.0 |
| Allyl heptanoate | 15.0 |
| Allyl amyl glycolate (Q) | 3.0 |
| Mefrosol (Q) | 15.0 |
| MDJ Super (Q) | 3.0 |
| Florocyclene (Q) | 4.0 |
| Hexyl salicylate (Q) | 5.0 |
| Iso-E-Super | 10.0 |
| Bangalol (Q) | 5.0 |
| Silvanone Supra (Q) | 5.0 |

### Example 3

| | % |
|---|---|
| Para-cresyl methyl ether | 6.0 |
| Camonal (Q) | 2.0 |
| Cyprisate (Q) | 3.0 |
| Isobornyl cyclohexanol | 0.5 |
| Florocyclene | 10.0 |
| Gamma decalactone | 6.0 |
| Ethyl vanillin 10% (DPG) | 0.5 |
| Silvanone (Q) | 2.5 |
| Isoamyl acetate | 4.0 |
| Herbanate (Q) | 1.0 |
| Manzanate (Q) | 1.0 |
| Amyl butyrate | 2.0 |
| Trans-2-hexenyl acetate | 1.0 |
| Ortholate (Q) | 8.0 |
| Herboxane (Q) | 8.0 |
| Phenoxyethanol | 8.0 |
| Phenoxyethyl isobutyrate | 10.0 |
| Beta-ionone | 10.0 |
| Karanal (Q) 10% (DPG) | 0.5 |

While the invention has been described with respect to preferred embodiments and examples, those skilled in the art will readily appreciate that various changes and/or modifications can be made to the invention without departing from the spirit or scope of the invention.

1. A hair-treatment composition comprising ammonia and an odor-reducing composition for counteracting malodorous amines wherein the odor-reducing composition comprises at least two odor-reducing materials selected from the group consisting of phenyl ethyl methyl ether, Cyprisate, Camonal and paracresyl methyl ether.

2. (canceled)

3. A hair-treatment composition as claimed in claim 1, wherein the odor-reducing material is present at 10 wt. % or above.

4. A hair-treatment composition as claimed in claim 3, wherein the odor-reducing material is present at 15 wt. % or above.

5. A hair-treatment composition as claimed in claim 4 wherein the odor-reducing material is present at 30 wt. % or above.

6. (canceled)

7. A hair-treatment composition as claimed in claim 1 wherein said composition is a hair colorant.

8. A hair-treatment composition as claimed in claim 1, wherein said composition is a bleaching composition for hair.

9. (canceled)

10. A hair-treatment composition as claimed in claim 1 wherein the ammonia is present in an amount from 0.5 wt. % to 5 wt. %.

11. A method of counteracting malodorous amines comprising adding to the source of the malodor a malodor counteracting amount of an odor-reducing composition comprising at least two odor-reducing materials selected from the group consisting of phenyl ethyl methyl ether, Cyprisate, Camonal and paracresyl methyl ether.

12. (canceled)

13. The method of claim 11, wherein said odor-reducing material is present in said odor-reducing composition at 10 wt. % or above.

14. The method of claim 11, wherein said odor-reducing material is present ins aid odor-reducing composition at 15 wt. % or above.

15. The method of claim 11, wherein said odor-reducing material is present in said odor-reducing composition at 30 wt. % or above.

16. The method of claim 11, wherein the malodorous amine comprises ammonia.

17. The method of claim 11, wherein the source of the malodour comprises a hair-treatment product.

18. The method of claim 17, wherein the hair-treatment product comprises a hair-colorant.

19. The method of claim 17, wherein the hair-treatment product is a bleaching composition for hair.
